(19) United States
(12) Patent Application Publication
(10) Pub. No.: US 2026/0130750 A1
(43) Pub. Date: May 14, 2026

(54) FLUID PRESSURE AMPLIFYING AND TRANSMITTING DEVICE FOR IMPROVING URINARY CONTINENCE FUNCTION

(71) Applicant: Tongji Hospital, Tongji Medical College, Huazhong University of Science and Technology, Wuhan City (CN)

(72) Inventors: Guanghui DU, Wuhan City (CN); Jinghao MU, Wuhan City (CN); Shengfei XU, Wuhan City (CN)

(73) Assignee: Tongji Hospital, Tongji Medical College, Huazhong University of Science and Technology, Wuhan City (CN)

(21) Appl. No.: 19/442,363

(22) Filed: Jan. 7, 2026

(30) Foreign Application Priority Data
Jan. 26, 2025 (CN) 202510121825.7

(51) Int. Cl. A61F 2/00

(52) U.S. Cl. CPC A61F 2/004; A61F 2250/0003; A61F 2250/0036; A61F 2250/0039

(57) ABSTRACT

A fluid pressure amplifying and transmitting device for improving urinary continence function is provided, which includes a first balloon, a second balloon, a first communicating tube and a second communicating tube, where the first balloon and the second balloon are both elastic bodies; the inner diameter of the first communicating tube is larger than that of the second communicating tube; the first communicating tube and the second communicating tube are both communicated between the first balloon and the second balloon, and the first balloon, the second balloon, the first communicating tube and the second communicating tube are filled with fluid; a first check valve with the flow direction from the first balloon to the second balloon is communicated on the first communicating tube; a second check valve with the flow direction from the second balloon to the first balloon is communicated on the second communicating tube.

### CROSS-REFERENCE TO RELATED APPLICATIONS

This application claims priority to Chinese Patent Application No. 202510121825.7, filed on Jan. 26, 2025, the contents of which are hereby incorporated by reference.

### TECHNICAL FIELD

The disclosure relates to the technical field of medical devices, and in particular to a fluid pressure amplifying and transmitting device for improving urinary continence function.

### BACKGROUND

Certain physiological pressures exist within organs and cavities of the living body, such as abdominal pressure, bladder pressure, rectal pressure, and urethral pressure. Under certain pathological conditions, elevated abdominal pressure may contribute to some disease states, such as stress urinary incontinence.

For stress urinary incontinence, treatment options generally include behavioral therapy, pharmacological therapy, and surgical therapy.

Behavioral therapy is mainly to exercise pelvic floor muscle ligament, and the effect is slow and inaccurate.

Pharmacological therapy may achieve an efficacy rate of 50%-60% in patients with mild to moderate symptoms; however, its therapeutic effect is unsatisfactory in patients with moderate to severe symptoms, and it is associated with significant side effects.

Surgical therapy is the primary treatment for stress urinary incontinence. Currently, conventional surgical procedures typically involve the implantation of a sling or an artificial urinary sphincter, but there are the following defects. First, the blind puncture technique used in sling implantation may cause physical damage, such as bladder perforation, injury to surrounding blood vessels, or vaginal injury. Second, the persistent compression exerted by the cuff or sling on urethral tissues may impair local blood circulation, subsequently leading to urethral atrophy and eventual recurrence of incontinence. Third, the structure of the artificial urinary sphincter is complex, and once implanted, it cannot be adjusted and carries a relatively high risk of mechanical failure.

### SUMMARY

The objective of the present disclosure is to provide a fluid pressure amplifying and transmitting device for improving urinary continence function. The device is capable of amplifying and transmitting the increased abdominal pressure during patient stress periods, along with the consequent pressure rise in the first balloon, to the second balloon located within urethral tissues, which results in an elevated urethral pressure in the patient, thereby achieving the effect of improving urinary continence function.

In order to achieve the above objective, the disclosure adopts the following technical scheme.

A fluid pressure amplifying and transmitting device for improving urinary continence function includes a first balloon, a second balloon, a first communicating tube and a second communicating tube;
where the first balloon and the second balloon are both elastic bodies;
an inner diameter of the first communicating tube is larger than an inner diameter of the second communicating tube;
the first communicating tube and the second communicating tube are both communicated between the first balloon and the second balloon, and the first balloon, the second balloon, the first communicating tube and the second communicating tube are filled with fluid;
a first check valve is communicated with the first communicating tube, and a flow direction of the first check valve is from the first balloon to the second balloon;
and a second check valve is communicated with the second communicating tube, and a flow direction of the second check valve is from the second balloon to the first balloon.

Further, a volume of the first balloon is larger than a volume of the second balloon.

Further, a volume ratio of the first balloon to the second balloon is 2-10.

Further, a diameter ratio of the first communicating tube to the second communicating tube is 2-20.

Further, the fluid is liquid.

Further, the liquid is water.

Further, a wall thickness of the first balloon is smaller than a wall thickness of the second balloon.

Further, both the first balloon and the second balloon are made of rubber materials.

Further, both the first communicating tube and the second communicating tube are made of PVC materials.

The disclosure has the following beneficial effects.

Firstly, the disclosure provides a fluid pressure amplifying and transmitting device for improving urinary continence function. The first balloon and the second balloon are implanted into the abdominal wall tissue space and urethral tissue of a patient respectively to amplify and transmit the increased abdominal pressure during patient stress periods, along with the consequent pressure rise in the first balloon, to the second balloon located within urethral tissues, which results in an elevated urethral pressure in the patient, thereby achieving the effect of improving urinary continence function.

Secondly, the disclosure provides a fluid pressure amplifying and transmitting device for improving urinary continence function. The balloon is implanted into the body tissue under direct vision, which not only ensures the positioning accuracy, but also avoids the risk of damage to tissues and organs such as bladder, vagina or surrounding great vessels caused by blind puncture.

Thirdly, the disclosure provides a fluid pressure amplifying and transmitting device for improving urinary continence function, and the balloon in the urethra is in a non-expanded state when the abdominal pressure is not increased, thereby preventing the blood vessels around the urethra from being damaged or even causing urethral atrophy due to long-term compression.

Fourthly, the disclosure provides a fluid pressure amplifying and transmitting device for improving urinary continence function, which includes balloons, communicating tubes and check valves. Different from the existing artificial urinary sphincter, the device has a simpler structure and may prevent complex mechanical component failures.

### BRIEF DESCRIPTION OF THE DRAWINGS

FIG. 1 is a schematic structural diagram of a fluid pressure amplifying and transmitting device for improving urinary continence function (the first balloon and the second balloon are in an initial state).

FIG. 2 is a schematic structural diagram of a fluid pressure amplifying and transmitting device for improving urinary continence function (the first balloon is squeezed and the second balloon is expanded).

FIG. 3 is a schematic structural diagram of a fluid pressure amplifying and transmitting device for improving urinary continence function implanted in the body.

FIG. 4 is a schematic structural diagram of the first balloon implanted into the body in a fluid pressure amplifying and transmitting device for improving urinary continence function of the present disclosure.

FIG. 5 is a schematic structural diagram of the second balloon implanted in a body in a fluid pressure amplifying and transmitting device for improving urinary continence function of the present disclosure.

FIG. 6 is a comparison diagram of the fluid pressure between the first balloon and the second balloon in a fluid pressure amplifying and transmitting device for improving urinary continence function (when the volumes of the first balloon and the second balloon are the same, and there is no first check valve on the first communicating tube and no second check valve on the second communicating tube).

FIG. 7 is a fluid pressure contrast diagram of the first balloon and the second balloon in a fluid pressure amplifying and transmitting device for improving urinary continence function (when the volume ratio of the first balloon to the second balloon is 5, and there is no first check valve on the first communicating tube and no second check valve on the second communicating tube).

FIG. 8 is a fluid pressure contrast diagram of a first balloon and a second balloon in a fluid pressure amplifying and transmitting device for improving urinary continence function of the present disclosure (when the volumes of the first balloon and the second balloon are the same, and the first communicating tube is communicated with the first check valve and the second communicating tube is not provided with the second check valve).

FIG. 9 is a comparison diagram of the fluid pressure between the first balloon and the second balloon in a fluid pressure amplifying and transmitting device for improving urinary continence function (when the volume ratio of the first balloon to the second balloon is 5, and the first communicating tube is communicated with the first check valve and the second communicating tube is not provided with the second check valve).

### DETAILED DESCRIPTION OF THE EMBODIMENTS

In order to make the objective, technical scheme and advantages of the disclosure more clear, the disclosure will be further explained in detail with the attached drawings and embodiments. It should be understood that the embodiments described here are only for explaining the disclosure and are not used to limit the disclosure.

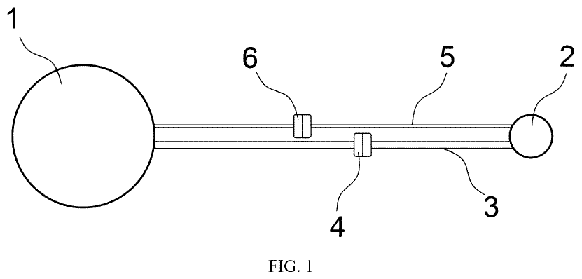

As shown in FIG. 1, a fluid pressure amplifying and transmitting device for improving urinary continence function includes a first balloon 1, a second balloon 2, a first communicating tube 3 and a second communicating tube 5.

The first balloon 1 and the second balloon 2 are both elastic bodies, and the volume of the first balloon 1 is larger than the volume of the second balloon 2.

Specifically, both the first balloon 1 and the second balloon 2 are made of rubber materials, and the volume ratio of the first balloon 1 to the second balloon 2 is 2-10.

Illustratively, in this embodiment, the volume ratio of the first balloon 1 to the second balloon 2 is 5.

The inner diameter of the first communicating tube 3 is larger than the inner diameter of the second communicating tube 5.

Specifically, the first communicating tube 3 and the second communicating tube 5 are both non-collapsed tubes, for example, made of PVC (polyvinyl chloride), and the diameter ratio of the first communicating tube 3 to the second communicating tube 5 is 2-20.

Illustratively, in this embodiment, the diameter ratio of the first communicating tube 3 to the second communicating tube 5 is 10.

The first communicating tube 3 and the second communicating tube 5 are both communicated between the first balloon 1 and the second balloon 2, and the first balloon 1 and the second balloon 2, as well as the first communicating tube 3 and the second communicating tube 5, are filled with fluid.

Specifically, the fluid is a liquid. The first balloon 1, the second balloon 2, and the first communicating tube 3 and the second communicating tube 5 are kept closed and free of bubbles.

Illustratively, in this embodiment, the liquid is water.

A first check valve 4 is communicated with the first communicating tube 3, and the flow direction of the first check valve 4 is from the first balloon 1 to the second balloon 2, and in the first communicating tube 3, the fluid flows along the first balloon 1 to the second balloon 2.

A second check valve 6 is communicated with the second communicating tube 5, and the flow direction of the second check valve 6 is from the second balloon 2 to the first balloon 1, and in the second communicating tube 5, the fluid flows along the second balloon 2 to the first balloon 1.

According to the above technical scheme, after the first balloon 1 and the second balloon 2 are implanted into the abdominal wall tissue space and urethral tissue of the patient respectively to amplify and transmit the increased abdominal pressure during patient stress periods, along with the consequent pressure rise in the first balloon, to the second balloon located within urethral tissues, which results in an elevated urethral pressure in the patient, thereby achieving the effect of improving urinary continence function. When the patient recovers from the stress period to the rest period, the pressure of the second balloon may be reduced to or close to the physiological level, thus avoiding lasting compression on the urethral tissue and further preventing the urethral tissue from atrophy.

As an application, the disclosure is capable of being used to eliminate urinary incontinence caused by the increase of abdominal pressure in patients, or to control urination in patients with urinary diversion.

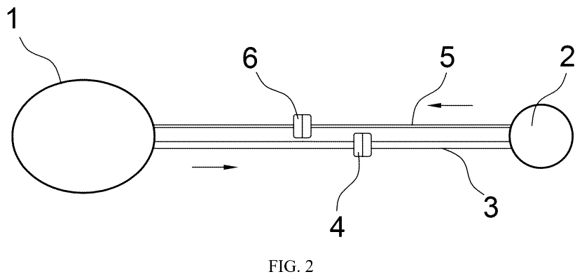

As shown in FIGS. 1-2, and the mechanism of the fluid pressure amplifying and transmitting device is as follows:
when the patient's abdominal pressure rises, the abdominal pressure acts on the first balloon 1 (the first balloon 1 will be squeezed) and the fluid in the first balloon 1. The fluid in the first balloon 1 is unidirectionally transmitted to the second balloon 2 through the first communicating tube 3, acts on the second balloon 2 (the second balloon 2 will expand) and acts on the tissues outside the second balloon 2, thus increasing the urethral pressure of the patient. At the same time, a small amount of fluid in the second balloon 2 is unidirectionally transmitted to the first balloon 1 through the second communicating tube 5.

With the disappearance of the patient's abdominal pressure rising state, the excess fluid in the second balloon 2 is unidirectionally transmitted to the first balloon 1 through the second communicating tube 5, and both the first balloon 1 and the second balloon 2 return to the initial state.

The increase of abdominal pressure in patients is generally due to cough, bending over, etc.

The volume of the first balloon 1 is larger than the volume of the second balloon 2, which is more conducive to storing sufficient fluid in the first balloon 1, and the fluid enters the second balloon 2 after being squeezed, so as to better realize the fluid pressure amplifying and transmitting between the first balloon 1 and the second balloon 2; the inner diameter of the first communicating tube 3 is larger than the inner diameter of the second communicating tube 5, so that the speed of the fluid in the first balloon 1 entering the second balloon 2 is faster than the speed of the fluid returning from the second balloon 2 to the first balloon 1 when the abdominal pressure of the patient increases due to cough and other phenomena, so as to better realize the fluid pressure amplifying and transmitting between the first balloon 1 and the second balloon 2.

In other embodiments, the wall thickness of the first balloon 1 is smaller than the wall thickness of the second balloon 2, which is more conducive to the extrusion of the first balloon 1 and the expansion of the second balloon 2, so as to better realize the fluid pressure amplifying and transmitting between the first balloon 1 and the second balloon 2.

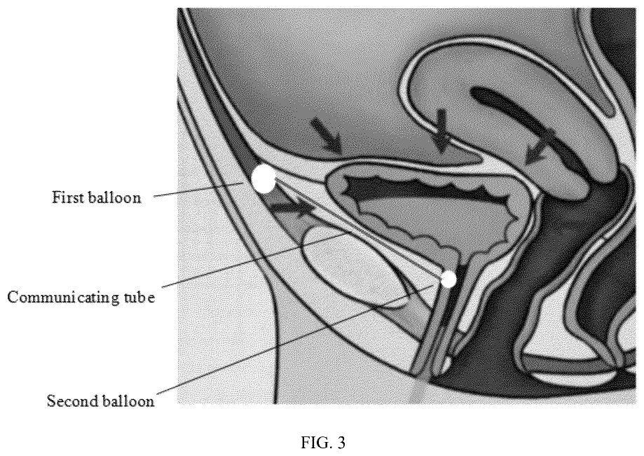

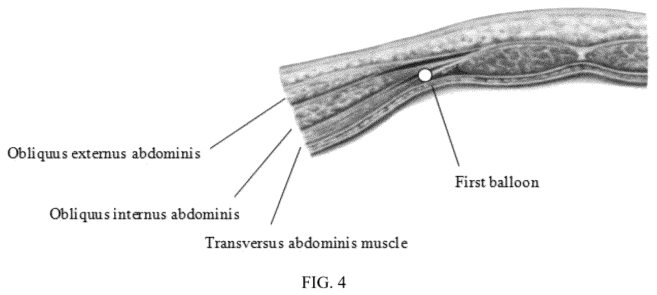

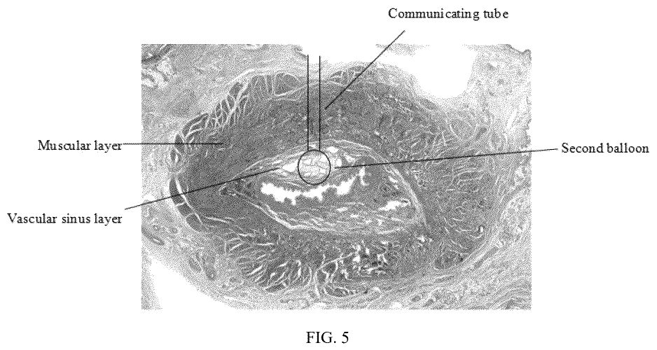

As shown in FIGS. 3-5, the operation process of implanting the fluid pressure amplifying and transmitting device into the body is as follows:
incising the adventitia and smooth muscle layer at the high pressure end of the urethra, placing the second balloon 2 in the urethral mucosa tissue with rich vascular sinuses between the smooth muscle of the urethra and the mucosal layer of the urethra, and then suturing the incision;
placing the first balloon 1 in any gap of the abdominal wall tissue, for example, in abdominal wall muscle, or between peritoneum and abdominal wall muscle, or between rectus abdominis and anterior sheath of rectus abdominis.

It should be noted that there are a large number of vascular sinus tissues with special shapes in the submucosa of the urethral high pressure area, so it is more in line with physiological conditions and has a bionic effect to place the second balloon 2 between the muscular layer of the urethra and the submucosa of the urethra; the first balloon 1 may also be placed in the abdominal cavity, but fixing is necessary by suture.

The following is a partial test result of the fluid pressure amplifying and transmitting device.

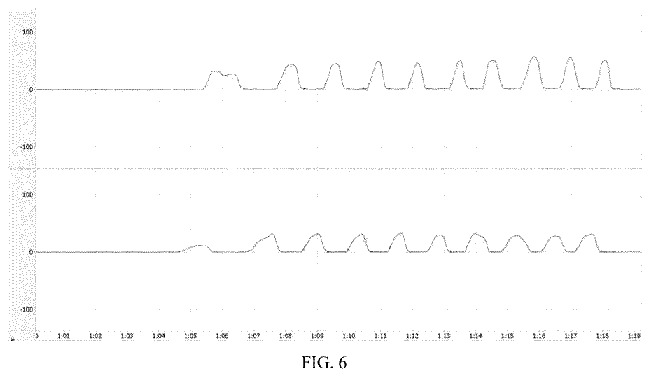

As shown in FIG. 6, the abscissa represents time, the ordinate represents fluid pressure, the lower curve represents fluid pressure in the first balloon, and the upper curve represents fluid pressure in the second balloon (the same below). The volumes of the first balloon 1 and the second balloon 2 are the same, and there is no first check valve 4 on the first communicating tube 3 and no second check valve 6 on the second communicating tube 5. When the fluid pressure in the first balloon 1 changes, the fluid pressure in the second balloon 2 changes accordingly, and the multiple of the pressure amplifying and transmitting is about 1.46

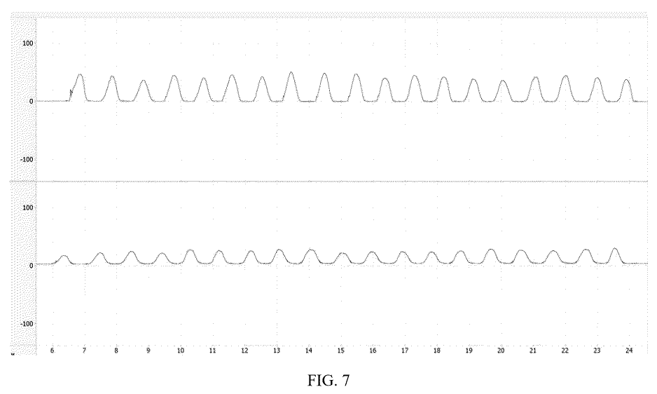

As shown in FIG. 7, the ratio of the volume of the first balloon 1 to the volume of the second balloon 2 is 5, and there is no first check valve 4 on the first communicating tube 3 and no second check valve 6 on the second communicating tube 5; when the fluid pressure in the first balloon 1 changes, the fluid pressure in the second balloon 2 changes accordingly, and the multiple of the pressure amplifying and transmitting is about 2.19.

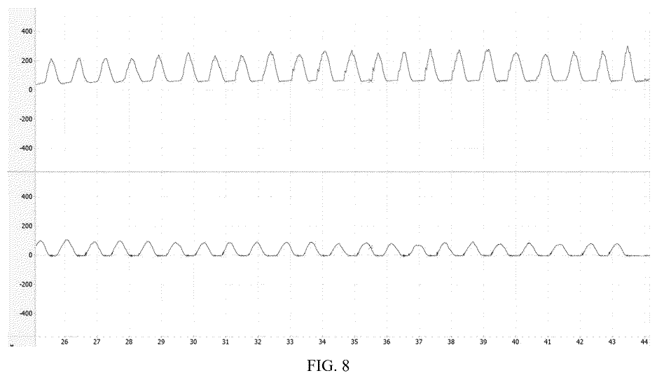

As shown in FIG. 8, the volumes of the first balloon 1 and the second balloon 2 are the same, and the first communicating tube 3 is communicated with the first check valve 4 and the second communicating tube 5 is communicated with the second check valve 6; when the fluid pressure in the first balloon 1 changes, the fluid pressure in the second balloon 2 changes accordingly, and the multiple of the pressure amplifying and transmitting is about 2.81.

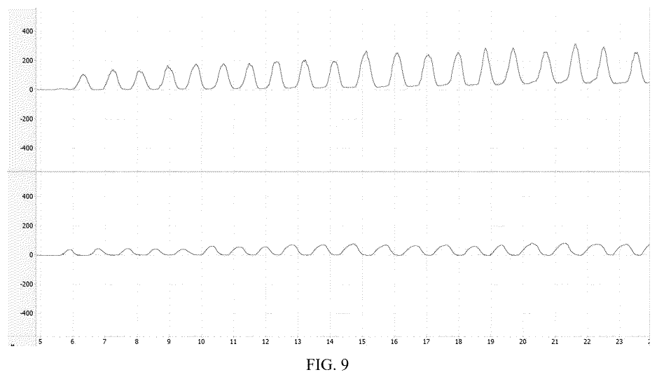

As shown in FIG. 9, the ratio of the volume of the first balloon 1 to the volume of the second balloon 2 is 5, and the first communicating tube 3 is communicated with the first check valve 4 and the second communicating tube 5 is communicated with the second check valve 6; when the fluid pressure in the first balloon 1 changes, the fluid pressure in the second balloon 2 changes accordingly, and the multiple of the pressure amplifying and transmitting is about 6.5.

According to the experimental results, whether there is a check valve on the first communicating tube 3 and the second communicating tube 5 has a great influence on the amplifying and transmitting of the fluid pressure amplifying and transmitting device; whether the volume of the first balloon 1 is larger than the volume of the second balloon 2 has a great influence on the amplifying and transmitting of the fluid pressure amplifying and transmitting device.

The present disclosure is not limited to the above specific embodiments. According to the disclosure of the present disclosure, ordinary technicians in the field may adopt other specific embodiments to implement the present disclosure. Therefore, any design that adopts the design structure and ideas of the present disclosure and makes some simple changes or modifications falls within the scope of protection of the present disclosure.

What is claimed is:

1. A fluid pressure amplifying and transmitting device for improving urinary continence function, comprising: a first balloon, a second balloon, a first communicating tube and a second communicating tube;
wherein the first balloon and the second balloon are both elastic bodies;
an inner diameter of the first communicating tube is larger than an inner diameter of the second communicating tube;
the first communicating tube and the second communicating tube are both communicated between the first balloon and the second balloon, and the first balloon, the second balloon, the first communicating tube and the second communicating tube are filled with fluid;
a first check valve is communicated with the first communicating tube, and a flow direction of the first check valve is from the first balloon to the second balloon;
and a second check valve is communicated with the second communicating tube, and a flow direction of the second check valve is from the second balloon to the first balloon.

2. The fluid pressure amplifying and transmitting device for improving the urinary continence function according to claim 1, wherein a volume of the first balloon is larger than a volume of the second balloon.

3. The fluid pressure amplifying and transmitting device for improving the urinary continence function according to claim 2, wherein a volume ratio of the first balloon to the second balloon is 2-10.

4. The fluid pressure amplifying and transmitting device for improving the urinary continence function according to claim 1, wherein a diameter ratio of the first communicating tube to the second communicating tube is 2-20.

5. The fluid pressure amplifying and transmitting device for improving the urinary continence function according to claim 1, wherein the fluid is liquid.

6. The fluid pressure amplifying and transmitting device for improving the urinary continence function according to claim 5, wherein the liquid is water.

7. The fluid pressure amplifying and transmitting device for improving the urinary continence function according to claim 1, wherein a wall thickness of the first balloon is smaller than a wall thickness of the second balloon.

8. The fluid pressure amplifying and transmitting device for improving the urinary continence function according to claim 1, wherein both the first balloon and the second balloon are made of rubber materials.

9. The fluid pressure amplifying and transmitting device for improving the urinary continence function according to claim 1, wherein both the first communicating tube and the second communicating tube are made of PVC materials.
